(19) United States
(12) Patent Application Publication
(10) Pub. No.: US 2025/0209580 A1
(43) Pub. Date: Jun. 26, 2025

(54) Noise Reduction in Retinal Images

(71) Applicant: Optos plc, Dunfermline (GB)

(72) Inventor: David Clifton, Dunfermline (GB)

(73) Assignee: Optos plc, Dunfermline (GB)

(21) Appl. No.: 18/989,247

(22) Filed: Dec. 20, 2024

(30) Foreign Application Priority Data
Dec. 22, 2023 (EP) 23 219 886.1

(51) Int. Cl. G06T 5/70; A61B 3/00; A61B 3/14; G06T 3/147; G06T 3/60; G06T 5/10; G06T 5/50; G06T 5/60; G06T 7/00; G06T 7/11; G06T 7/37; G06T 7/40; G06V 10/774

(52) U.S. Cl. CPC G06T 5/70; A61B 3/0025; A61B 3/145; G06T 3/147; G06T 3/60; G06T 5/10; G06T 5/50; G06T 5/60; G06T 7/0016; G06T 7/11; G06T 7/37; G06T 7/40; G06V 10/774; G06T 2207/10064; G06T 2207/20081; G06T 2207/20216; G06T 2207/30041; G06T 2207/30201

(57) ABSTRACT

A computer-implemented method of processing a sequence of images of a region of a retina to generate an averaged image of the region, comprising: determining, for each combination of a reference image selected from the sequence of images and a respective comparison image being an image from remaining images in the sequence, a respective offset between the reference image and the respective comparison image; comparing each offset with an threshold to determine whether the offset is smaller than the threshold; selecting the respective comparison image in each combination for which the respective offset has been determined to be smaller than the threshold; and using the selected comparison images to generate the averaged image of the region, wherein the threshold is such that the averaged image shows more texture features in the region of the retina than a reference averaged image generated from the images in the sequence of images.

### FIELD

Example aspects herein generally relate to techniques for suppressing noise in images of a retina of an eye and, more particularly, to the processing of a sequence of images of a region of a retina to generate a de-noised image of the region.

### BACKGROUND

Retinal imaging of various different modalities is widely used to detect pathological and age-related physiological changes in the retina of the eye. In all cases, successful detection of such changes requires retinal images to be in focus and to have as little noise and as few artifacts as possible. However, for patient safety, retinal imaging devices generally use low-power illumination sources, which can result in a lower signal-to-noise ratio (SNR) than might be achieved in the imaging other tissues. Furthermore, noise is inherent in some imaging techniques. For example, optical coherence tomography (OCT) images tend to contain speckle noise, which can reduce contrast and make it difficult to identify boundaries between strongly scattering structures. Fundus autofluorescence (FAF) images are derived from detections of low luminance level fluorescence, and often have a poor SNR owing to a combination of photon noise and amplifier (readout) noise.

Noise may be suppressed by adapting the image capture process, for example by using an averaging technique whereby multiple image frames are recorded and registered with respect to each other, with the registered images then being averaged to produce a de-noised image in which the noise from the component images averages out to a lower level. Noise may also be suppressed by post-capture image processing techniques, for example by using a convolutional neural network (CNN) or other supervised machine learning algorithm, which has been trained using averaged (de-noised) images to remove noise from a captured retinal image. However, both these approaches tend to result in valuable low-contrast anatomical features, including texture associated with the retina, being suppressed or absent in the final de-noised image. It would therefore be desirable to provide techniques for producing de-noised retinal images that retain the texture associated with the retina.

### SUMMARY

There is provided, in accordance with a first example aspect herein, a computer-implemented method of processing a sequence of images of a region of a retina of an eye to generate an averaged image of the region, the method comprising: determining, for each combination of a reference image selected from the sequence of images and a respective comparison image being an image from remaining images in the sequence, a respective offset between the reference image and the respective comparison image; comparing each determined offset with an offset threshold to determine whether the offset is smaller than the offset threshold; selecting the respective comparison image in each combination for which the respective offset between the reference image and the respective comparison image has been determined to be smaller than the offset threshold; and using the selected comparison images to generate the averaged image of the region, wherein the offset threshold is such that, where the sequence of images comprises at least one image which is offset from the reference image by an offset greater than the threshold, and images that are offset from the reference image by respective offsets that are smaller than the threshold, the averaged image shows more texture which is indicative of a structure in the first region of the retina than a reference averaged image generated from the images in the sequence of images.

There is provided, in accordance with a second example aspect herein, a computer-implemented method of training a machine learning algorithm to filter noise from retinal images, the method comprising: generating ground truth training target data by processing each sequence of a plurality of sequences of retinal images to generate a respective averaged retinal image, wherein each averaged retinal image is generated in accordance with the computer-implemented method of the first example aspect; generating training input data by selecting a respective image from each of the sequences of images; and using the ground truth training target data and the training input data to train the machine learning algorithm to filter noise from retinal images.

There is also provided, in accordance with a third example aspect herein, a computer program comprising computer-readable instructions that, when executed by at least one processor, cause the at least one processor to perform the method of at least one of the first example aspect or the second example aspect set out above. The computer program may be stored on a non-transitory computer-readable storage medium (such as a computer hard disk or a CD, for example) or carried by a computer-readable signal.

There is also provided, in accordance with a fourth example aspect herein, a data processing apparatus arranged to process a sequence of images of a region of a retina of an eye to generate an averaged image of the region, the data processing apparatus comprising at least one processor and at least one memory storing computer-readable instructions that, when executed by the at least one processor, cause the at least one processor to: determine, for each combination of a reference image selected from the sequence of images and a respective comparison image being an image from remaining images in the sequence, a respective offset between the reference image and the respective comparison image; compare each determined offset with an offset threshold to determine whether the offset is smaller than the offset threshold; select the respective comparison image in each combination for which the respective offset between the reference image and the respective comparison image has been determined to be smaller than the offset threshold; and use the selected comparison images to generate the averaged image of the region, wherein the offset threshold is such that, where the sequence of images comprises at least one image which is offset from the reference image by an offset greater than the threshold, and images that are offset from the reference image by respective offsets that are smaller than the threshold, the averaged image shows more texture which is indicative of a structure in the first region of the retina than a reference averaged image generated from the images in the sequence of images.

There is also provided, in accordance with a fifth example aspect herein, an ophthalmic imaging system comprising an ophthalmic imaging device arranged to acquire a sequence of images of a region of a retina of an eye, and a data processing apparatus according to the fourth example aspect, which is arranged to process the sequence of images acquired by the ophthalmic imaging device to generate an averaged image of the region of the retina.

### BRIEF DESCRIPTION OF THE DRAWINGS

Example embodiments will now be explained in detail, by way of non-limiting example only, with reference to the accompanying figures described below. Like reference numerals appearing in different ones of the figures can denote identical or functionally similar elements, unless indicated otherwise.

FIG. 1 is a schematic illustration of an ophthalmic imaging system 100 comprising an ophthalmic imaging device 10 and a data processing apparatus 60 according to example embodiments herein.

FIG. 2 is a schematic illustration of an example process by which the data processing apparatus 60 may processes a sequence of images 20 of a region of a retina of an eye to generate an averaged image 70 of the region.

FIG. 3 is a schematic illustration of an example implementation of the data processing apparatus 60 in programmable signal processing hardware 300.

FIG. 4 is a flow diagram illustrating a method according to a first example embodiment, by which the data processing apparatus 60 processes a sequence of images of a region of a retina to generate an averaged image of the region.

FIG. 5 shows an example of a FAF image, and a 1.3-pixel displacement at an edge of the image caused by a 0.3-degree rotation about the centre of the image.

FIG. 6A shows an example of a FAF image.

FIG. 6B shows an averaged FAF image, which has been obtained by averaging the FAF image shown in FIG. 6A with another FAF image which is rotationally offset from the image of FIG. 6A by 4.5 degrees.

FIG. 6C shows an averaged FAF image, which has been obtained by averaging the FAF image shown in FIG. 6A with another FAF image which has a negligible rotational offset relative to the image of FIG. 6A.

FIG. 7 is a schematic illustration of a segmentation of a second sequence of images 15 to generate the sequence of images 20.

FIG. 8 is a flow diagram illustrating a method according to a second example embodiment, by which the data processing apparatus 60 processes a sequence of images of a region of a retina to generate an averaged image of the region.

FIG. 9A shows an example FAF image acquired by the ophthalmic imaging device 10.

FIG. 9B shows an example of the averaged image 70 obtained by averaging 10 FAF images, including the FAF image of FIG. 9A, that have been selected from a sequence of FAF images in accordance with the method of the second example embodiment.

FIG. 10 is a flow diagram illustrating a method according to a third example embodiment, by which the data processing apparatus 60 trains a machine learning algorithm to filter noise from retinal images.

FIG. 11 is a flow diagram illustrating the method by which the processor 320 generates each of the averaged retinal images from the respective sequence of retinal images in the third example embodiment.

### DETAILED DESCRIPTION OF EXAMPLE EMBODIMENTS

### First Example Embodiment

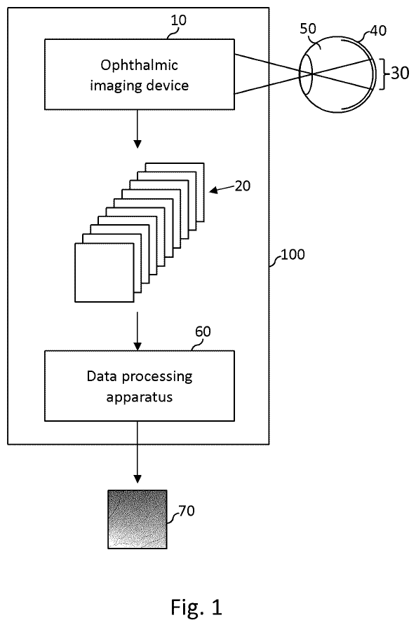

FIG. 1 is a schematic illustration of an ophthalmic imaging system 100 according to an example embodiment, which comprises an ophthalmic imaging device 10, which is arranged to acquire a sequence of images 20 of a common region 30 of a retina 40 of an eye 50. The ophthalmic imaging system 100 further comprises a data processing apparatus 60, which is arranged to process images from the sequence of images 20 to generate an averaged image 70 of the region 30 of the retina 40.

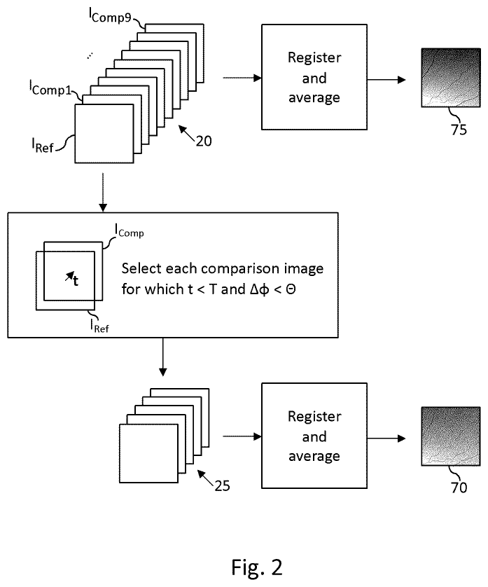

As illustrated schematically in FIG. 2, the data processing apparatus 60 is arranged to generate the averaged image 70 by firstly selecting images from the sequence of images 20 according to one or more predefined criteria. These images may be selected by firstly determining, for each combination of a reference image, I_{Ref}, which has been selected from the sequence of images 20 and a respective image from the sequence 20 being considered for the selection (herein referred to as a comparison image, I_{Comp}), a respective offset between the reference image I_{Ref} and the respective comparison image I_{Comp}. Each determined offset may then be compared with an offset threshold to determine whether the offset is smaller than the offset threshold. By way of an example, the one or more predefined criteria may include a criterion that the magnitude t=|t| of a determined translational offset t between the comparison image I_{Comp} and the reference image I_{Ref} is less that a predetermined translational offset threshold T. Additionally or alternatively, the one or more predefined criteria may include a criterion that the magnitude of a determined rotational offset (i.e. a relative rotation) Δϕ between the comparison image I_{Comp} and the reference image I_{Ref} is less than a predetermined rotational offset threshold Θ. The selected comparison images 25 are registered with respect to one another by the data processing apparatus 60, and the data processing apparatus 60 then calculates an average of the registered images, for example such that the respective pixel value of each pixel in the resulting averaged image 70 is a mean (e.g. arithmetic mean) of the pixel values of correspondingly located pixels in the registered selected images 25 that correspond to respective common location in the region 30 of the retina 40. The data processing apparatus 60 may alternatively calculate the average of the registered images such that the respective pixel value of each pixel in the resulting averaged image 70 is a weighted average of the pixel values of correspondingly located pixels in the registered selected images 25 that correspond to respective common location in the region 30 of the retina 40, for example.

In this process (examples of which are described below), in a case where the sequence of images 20 comprises at least one image which is offset from the reference image I_{Ref} by an offset greater than the threshold, and images that are offset from the reference image I_{Ref} by respective offsets that are smaller than the threshold, the one or more predefined criteria result in images being selected in such a way that the resulting averaged image 70 shows more texture than a reference averaged image 75 generated from some or all the images in the sequence of images 20, without these images having been selecting in the way herein described. In this context, the texture is an anatomical texture associated with the retina, which is indicative of an anatomical structure in the region 30 of the retina 40. For example, where the images acquired by the ophthalmic imaging device 10 are fundus autofluorescence (FAF) images, as in the present example embodiment, the structure may be defined by a spatial distribution of fluorophores across the region 30 of the retina 40. As another example, where the images acquired by the ophthalmic imaging device 10 are OCT images, the structure may comprise a physical structure of one or more layers of the retina 40 in the region 30 of the retina 40. As a further example, where the images acquired by the ophthalmic imaging device 10 are reflectance images (e.g. red, blue or green reflectance images) of the region 30 of the retina 40, the structure may comprise the upper surface of the retina in the region 30, such that the texture is a physical texture of the surface that reflects the topography of the surface.

Furthermore, the averaged image 70 tends to have a higher SNR than a single image from the sequence of images 20, owing to the noise components in the selected images 25 partially cancelling each other out because of the selected images 25 being averaged. The averaged image 70 generated by the data processing apparatus 60 of the example embodiment is thus a de-noised image of the region 30 of the retina 40, in which more of the texture is retained than in a conventional de-noised image (e.g. image 75) which has been generated by averaging images of the sequence 20 that have not been selected according to one or more of the criteria described herein. The image processing techniques described herein may thus produce de-noised and anatomically correct retinal images, in which important clinical data is preserved. Such de-noised images are valuable not only to clinicians for assessing the health of the retina 40 but also for the training of artificial intelligence (AI) denoising algorithms to suppress noise whilst reducing or avoiding a loss of texture in the de-noised image that often results from the use of conventionally trained AI denoising algorithms, as discussed in more detail below.

Suppression of noise whilst retaining texture in the averaged image 70 requires the processing of the sequence of images 20 to take into account the following classes of imperfections in the image acquisition process and their effects on the acquired images. Firstly, the reference image I_{Ref} and a comparison image I_{Comp} may be related by an affine or a non-affine transformation (e.g. a translation, a rotation or a warping in one or more directions), which may be caused by a movement of the retina during acquisition of the images which is in a direction normal to the imaging axis, a rotation of the retina about the imaging axis or a movement of the retina which occurs as a result of a change in gaze direction of the eye. Secondly, an acquired image may have a non-affine warping within it, which may be caused by an eye movement, such as a rapid horizontal saccade, which occurs during capture of the image. Thirdly, there may be one or more localised regions or patches of micro-warping (micro-misalignment) within some of the images, which may be caused by variations in the imaging system (e.g. a beam scanning system therein comprising a polygonal mirror or other scanning element) and consequent light path variations between captures of different images. The light path variation may, for example, be caused by deviations from scan linearity and/or mirror imperfections, among other factors.

The ophthalmic imaging device 10 may, as in the present example embodiment, be any kind of FAF imaging device well-known to those versed in the art (e.g. a fundus camera, a confocal scanning laser ophthalmoscope (SLO) or an ultra-widefield imaging device such as the Daytona™ from Optos™), which is arranged to capture a sequence of FAF images of the same region of the retina 30. FAF imaging devices often use short to medium wavelength visible excitation, and collect emissions within a predefined spectral band (e.g. within a wavelength range of 500 to 750 nm) to form a brightness map reflecting the distribution of lipofuscin, which is a dominant fluorophore located in the retinal pigment epithelium (RPE). However, other excitation wavelengths, such as near-infrared, can be used to detect additional fluorophores, such as melanin. FAF is useful in the evaluation of a variety of diseases involving the retina and RPE.

The form of the ophthalmic imaging device 10 is not so limited, however. In other example embodiments, the ophthalmic imaging device 10 may, for example, take the form of an OCT imaging device, such as a swept-source OCT (SS-OCT) imaging device or a spectral domain OCT (SD-OCT) imaging device, which is arranged to acquire OCT images, e.g. B-scans, C-scans and/or en-face OCT images, of the region 30 of the retina 40. In yet other example embodiments, the ophthalmic imaging device 10 may comprise an SLO, a fundus camera or the like, which is arranged to capture reflectance images (e.g. red, green or blue images) of the region 30 of the retina 40.

Although the ophthalmic imaging device 10 is shown in FIG. 1 to acquire a sequence of 10 images, this number has been given by way of an example only. The number of images in the sequence of images 20 is not limited to 10, and there may be more or fewer images in the sequence of images 20. It is also noted that the region 30 is a portion of the retina 40 which lies within a field of view (FoV) of the ophthalmic imaging device 10, and may be smaller than a portion of the retina 40 that is spanned by the FoV. As described in more detail below, the region 30 may be a segment of a larger region of the retina 40 which the ophthalmic imaging device 10 may be arranged to capture images of, where each image spans the field-of-view of the ophthalmic imaging device 10. Each image in the sequence of images 20 may therefore be a segment (e.g. a rectangular image tile) of a respective larger retinal image of the retina 40 which has been captured by the ophthalmic imaging device 10.

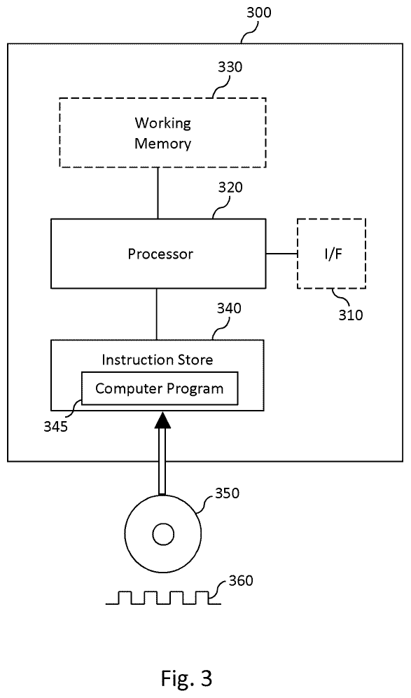

The data processing apparatus 60 may be provided in any suitable form, for example as a programmable signal processing hardware 300 of the kind illustrated schematically in FIG. 3. The programmable signal processing apparatus 300 comprises a communication interface (I/F) 310, for receiving the sequence of images 20 from the ophthalmic imaging device 10, and outputting the averaged image 70. The signal processing hardware 300 further comprises a processor (e.g. a Central Processing Unit, CPU, and/or a Graphics Processing Unit, GPU) 320, a working memory 330 (e.g. a random-access memory) and an instruction store 340 storing a computer program 345 comprising the computer-readable instructions which, when executed by the processor 320, cause the processor 320 to perform the various functions of the data processing apparatus 60 described herein. The working memory 330 stores information used by the processor 320 during execution of the computer program 345, such as the reference image I_{Ref}, the comparison images I_{Comp1} to I_{Comp9}, the calculated image offsets, image offset thresholds, selected comparison images 25, determined degrees of similarity, the similarity thresholds and the various intermediate processing results described herein. The instruction store 340 may comprise a ROM (e.g. in the form of an electrically erasable programmable read-only memory (EEPROM) or flash memory) which is pre-loaded with the computer-readable instructions. Alternatively, the instruction store 340 may comprise a RAM or similar type of memory, and the computer-readable instructions of the computer program 345 can be input thereto from a computer program product, such as a non-transitory, computer-readable storage medium 350 in the form of a CD-ROM, DVDROM, etc. or a computer-readable signal 360 carrying the computer-readable instructions. In any case, the computer program 345, when executed by the processor 320, causes the processor 320 to perform the functions of the data processing apparatus 60 described herein. Thus, the data processing apparatus 60 of the example embodiment may comprise a computer processor 320 (or two or more such processors) and a memory 340 (or two or more such memories) storing computer-readable instructions which, when executed by the processor(s), cause the processor(s) to process the sequence of images 20 to generate the averaged image 70 of the region 30 of the retina 40 as herein described.

It should be noted, however, that the data processing apparatus 60 may alternatively be implemented in non-programmable hardware, such as an ASIC, an FPGA or other integrated circuit dedicated to performing the functions of the data processing apparatus 60 described herein, or a combination of such non-programmable hardware and programmable hardware as described above with reference to FIG. 3. The data processing apparatus 60 may be provided as a stand-alone product or as part of the ophthalmic imaging system 100.

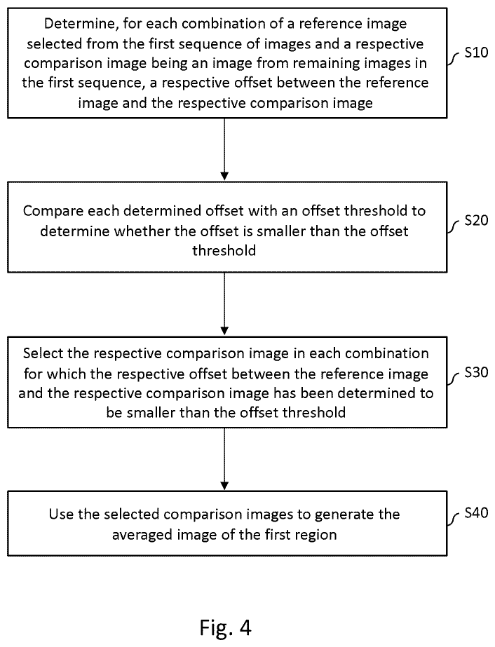

A process by which the data processing apparatus 60 of the present example embodiment processes the sequence of images 20 to generate the averaged image 70 of the region 30 of the retina 40 will now be described with reference to FIG. 4.

In process S10 of FIG. 4, the processor 320 of the data processing apparatus 60 determines, for each combination of a reference image I_{Ref}, which has been selected from the sequence of images 20, and a respective comparison image I_{Comp} from remaining images in the sequence of images 20, a respective offset between the reference image I_{Ref} and the respective comparison image I_{Comp}. The respective offset may comprise a rotation of the comparison image I_{Comp} in the combination relative to the reference image I_{Ref} in the combination and/or a translation of the comparison image I_{Comp} in the combination relative to the reference image I_{Ref} in the combination.

The reference image I_{Ref} may be selected from the sequence of images 20 in a variety of different ways. The reference image I_{Ref} may, for example, be the image that appears at a predetermined position in the sequence of images 20, for example at the beginning, at the end, or preferably at an intermediate position in the sequence of images 20 (e.g. the middle of the sequence). The reference image I_{Ref} may alternatively be selected at random from the images in the sequence of images 20. However, to more reliably achieve as large a number of selected images 25 as possible, and thus make noise suppression in the averaged image 70 (which is derived from the selected images 25) more effective, the reference image I_{Ref} is preferably selected to be an image from among the images in the sequence of images 20 for which the number of remaining images in the sequence that are offset from the reference image I_{Ref} by an amount less than the predetermined offset threshold is highest (or equal highest). The reference image I_{Ref} may, for example, be selected by determining, for any image chosen from the sequence of images 20 to serve as a base image, a respective translational offset between the base image and each image of the remaining images in the sequence of images 20, for example by determining the respective location of a peak in a cross-correlation calculated between the base image and each image of the remaining images. The translational offsets determined in this way may be represented as a set of points in a two-dimensional plot. For each point in this plot, the respective number of remaining points that are within a circle of radius T centred on the point is determined. Once this has been done for all the points in the plot, a point having the highest (or equal highest) number of remaining points within the circle centred on it is selected. The image from the sequence of images, whose offset from the base image is represented by the selected point, is then selected as the reference image I_{Ref}.

The processor 320 thus determines, for each image of the remaining images in the sequence 20 (i.e. other than the base image), a respective count by determining a respective translational offset between the image and every other image in the sequence, comparing the determined translational offsets with the offset threshold T to identify every other image in the sequence having a translational offset from the image which is smaller than the translational threshold T, and counting the identified images to determine the respective count. The processor 320 then uses the determined counts to identify, as the reference image I_{Ref}, an image of the remaining images in the sequence 20 for which the determined count is highest (or equal highest).

The processor 320 may, as in the present example embodiment, determine in process S10 of FIG. 4, for each combination of the reference image I_{Ref} and a respective comparison image I_{Comp} from some or all the remaining images in the sequence of images 20, a respective translational offset between the reference image I_{Ref} and the respective comparison image I_{Comp}, and a respective rotational offset between the reference image I_{Ref} and the respective comparison image I_{Comp}. However, in other example embodiments, the processor 320 may determine, for each combination of the reference image I_{Ref} and a respective comparison image I_{Comp} from remaining images in the sequence of images 20, either a respective translational offset between the reference image I_{Ref} and the respective comparison image I_{Comp}, or a respective rotational offset between the reference image I_{Ref} and the respective comparison image I_{Comp} (but not both).

The translational and rotational offsets between images may be determined using various techniques as described herein or well-known to those versed in the art. For example, in cases where relative rotations between images in the sequence of images 20 are negligible, the respective translational offset t between the reference image I_{Ref} and each comparison image I_{Comp} may be determined by calculating a cross-correlation between the reference image I_{Ref} and the comparison image I_{Comp}, with the location of a peak in the calculated cross-correlation being used to determine the respective translational offset t. As the self-similarity of the images in the sequence of images 20 may result in relatively broad peaks in the calculated cross-correlations, the images may be pre-processed prior to being cross-correlated (using an edge detector, for example), to sharpen the peaks and thus allow the translational offset t to be determined more precisely.

As an alternative, the respective translational offset t between the reference image I_{Ref} and each comparison image I_{Comp} may be determined by firstly calculating a normalized cross-power spectrum of the reference image I_{Ref} and the comparison image I_{Comp}, which may be expressed as F_{Ref}F_{Comp}*/|F_{Ref}F_{Comp}*|, where F_{Ref} is a discrete Fourier transform of the reference image I_{Ref}, and F_{Comp}* is the complex conjugate of a discrete Fourier transform of the comparison image I_{Comp}. An inverse Fourier transform of the normalized cross-power spectrum may then be calculated to provide a delta function which has a maximum value at (Δx, Δy), where the comparison image I_{Comp} is shifted by Δx pixels in an x-axis direction relative to the reference image I_{Ref}, by Δy pixels in a y-axis direction relative to the reference image I_{Ref}.

The cross-correlation approach described above may, as in the present example embodiment, be extended to allow for relative rotations between the images in the sequence of images 20 by determining a respective translational offset t between the reference image I_{Ref} and each of a plurality of rotated versions of the comparison image I_{Comp}. This may be done by calculating a respective cross-correlation between the reference image I_{Ref} and each rotated version of the comparison image I_{Comp}. By way of an example and without limitation, each comparison image I_{Comp} (with or without the pre-processing mentioned above) may be rotated between −6 degrees and +6 degrees in increments of 0.1 degrees to generate 120 rotated versions of each comparison image I_{Comp}, each of which (along with the original (unrotated) comparison image I_{Comp}) is cross correlated with the reference image I_{Ref}. The angular range of −6 degrees to +6 degrees, and the increments of 0.1 degrees, have been given by way of example only, and other ranges and/or angular increments may alternatively be used. The rotation (if any) applied to the image that is cross-correlated with the reference image I_{Ref} to produce a cross-correlation having the highest peak of the peaks in the 121 calculated cross-correlations may be taken to provide an indication of the rotational offset between the reference image I_{Ref} and the comparison image I_{Comp}, while the location of the highest cross-correlation peak may be taken to provide an indication of the translational offset t between the reference image I_{Ref} and the comparison image I_{Comp}.

The rotational offset Δϕ and the translational offset t between the reference image I_{Ref} and the comparison image I_{Comp}, may alternatively be calculated in the Fourier domain. This approach relies on two properties of the Fourier transform. First, the frequency spectrum is always centred on the origin, regardless of any shifts in the underlying image. Second, the rotation of an image will always result in a corresponding rotation in its Fourier transform. In the alternative approach, the reference image I_{Ref} and the comparison image I_{Comp} are transformed to polar coordinates. An image I(x, y) is thus transformed to I(r, ϑ), and the discrete Fourier transform of the image, F(u, v), becomes F(ρ, ϕ), so that

$${F}_{Ref} ( ρ , ϕ ) = \int \int {I}_{R e f} ( r , θ ) {e}^{- 2 π i ρ r cos ( θ - ϕ )} r d r d θ$$

Given an angle β, such that I_{Comp} is the rotation of I_{Ref} by β in the clockwise direction, it can be seen that I_{Comp}(r, ϑ)=I_{Ref}(r,ϑ+β). This means that

$${F}_{Comp} ( ρ , ϕ ) = \int \int {I}_{R e f} ( r , θ + β ) {e}^{- 2 π i ρ r cos ( θ - ϕ )} r d r d θ$$

which is equivalent to

$${F}_{Comp} ( ρ , ϕ ) = \int \int {I}_{R e f} ( r , θ ) {e}^{- 2 π i ρ r cos ( θ - ( ϕ + β ) )} r d r d θ$$

so that

$${F}_{Comp} ( ρ , ϕ ) = {F}_{Ref} ( ρ , ϕ + β ) .$$

These two properties separate the rotational and translational components of image registration so that they can be solved independently. For a set of images that are both shifted and rotated with respect to each other, the rotational shift between the images can be isolated by taking their Fourier transform. The magnitude of the frequency components created by this Fourier transform are treated as if they are the pixel values for a new set of images that only require rotational registration. Once the rotational correction is found, it is applied to the underlying image to be registered. The translational parameters are then found using the same algorithm. The respective rotational offset Δϕ between the reference image I_{Ref} and the respective comparison image I_{Comp} may thus be determined calculating an inverse Fourier transform of a normalized cross-power spectrum of polar transformations of the reference image I_{Ref} and the respective comparison image I_{Comp}. Further details of this approach are provided in the article titled “Medical Image Registration Using the Fourier Transform” by J. Luce et al., International Journal of Medical Physics, Clinical Engineering and Radiation Oncology, 2024, 3, pages 49-55 (February 2014), the contents of which are incorporated herein by reference in their entirety.

Other intensity-based algorithms may alternatively be used to determine offsets between the reference image I_{Ref} and the comparison images I_{Comp} in process S10 of FIG. 4, including those based on similarity measures other than cross-correlation, for example mutual information, sum of squared intensity differences, or ratio image uniformity (among others).

Referring again to FIG. 4, in process S20, the processor 320 compares each determined offset with an offset threshold to determine whether the offset is smaller than the offset threshold. The processor 320 may, as in the present example embodiment, compare each translational offset t with the translational offset threshold T to determine whether the translational offset t is smaller than the translational offset threshold T. The processor 320 may, as in the present example embodiment, also compare each rotational offset Δϕ with the rotational offset threshold Θ to determine whether the rotational offset Δϕ is smaller than the rotational offset threshold Θ. In other example embodiments, where either a respective translational offset between the reference image I_{Ref} and the respective comparison image I_{Comp}, or a respective rotational offset between the reference image I_{Ref} and the respective comparison image I_{Comp}, is determined by the processor 320 for each combination of the reference image I_{Ref} and a respective comparison image I_{Comp} from remaining images in the sequence of images 20 (in process S10 of FIG. 4), the processor 320 may either determine whether the translational offset t is smaller than the translational offset threshold T, or whether the rotational offset Δϕ is smaller than the rotational offset threshold Θ in process S20 of FIG. 4, as the case may be. The setting of the offset threshold values T and Θ is described below.

In process S30 of FIG. 4, the processor 320 selects the respective comparison image I_{Comp} in each combination for which the respective offset between the reference image I_{Ref} and the respective comparison image I_{Comp} has been determined to be smaller than the offset threshold. For any combination for which the respective offset between the reference image I_{Ref} and the respective comparison image I_{Comp} has been determined to not be smaller than the offset threshold, the processor 320 does not select the comparison image I_{Comp} in that combination.

The processor 320 may, as in the present example embodiment, select the respective comparison image I_{Comp} in each combination for which the respective translational offset t has been determined to be smaller than the translational offset threshold T, and for which the respective rotational offset Δϕ has been determined to be smaller than the rotational offset threshold Θ in process S20 of FIG. 4. In other example embodiments, where either a respective translational offset between the reference image I_{Ref} and the respective comparison image I_{Comp}, or a respective rotational offset between the reference image I_{Ref} and the respective comparison image I_{Comp}, is determined by the processor 320 for each combination of the reference image I_{Ref} and a respective comparison image I_{Comp} from remaining images in the sequence of images 20 (in process S10 of FIG. 4), the processor 320 may select the respective comparison image I_{Comp} in each combination for which either the respective translational offset t has been determined to be smaller than the translational offset threshold T, or the respective rotational offset Δϕ has been determined to be smaller than the rotational offset threshold Θ in process S20 of FIG. 4, as the case may be.

In process S40 of FIG. 4, the processor 320 uses the selected comparison images 25 to generate the averaged image 70 of the region 30 of the retina 40. The processor 320 may, as in the present example embodiment, use the selected comparison images 25 (and optionally also the reference image I_{Ref}) to generate the averaged image 70 of the region 30 by registering the selected images 25 (and optionally also the reference image I_{Ref}) with respect to each other, and averaging the registered images to generate the averaged image 70 of the first region 30, for example such that the respective pixel value of each pixel in the averaged image 70 is a mean (e.g. an arithmetic mean) of the pixel values of correspondingly located pixels in the registered images that correspond to respective common location in the region 30 of the retina 40. The processor 320 may alternatively average the registered images to generate the averaged image 70 of the first region 30 such that the respective pixel value of each pixel in the averaged image 70 is a weighted average of the pixel values of correspondingly located pixels in the registered selected images 25 that correspond to respective common location in the region 30 of the retina 40, for example.

The offset thresholds T and Θ are selected such that, in case the sequence of images 20 comprises at least one image which is offset from the reference image I_{Ref} by a translational offset t greater than the translational offset threshold T and/or by a rotational offset Δϕ greater than the rotational offset threshold Θ, and images whose respective translational and rotational offsets from the reference image I_{Ref} are smaller than T and Θ, respectively, the averaged image 70 shows more texture which is indicative of a structure in the region 30 of the retina 40 than the reference averaged image 75 generated from the images in the sequence of images 20.

As the images being compared in process S20 of FIG. 4 often differ by a non-affine transformation that tends to produce a deformation which increases with increasing offset (whether translational or rotational) between the images, a higher correlation between the content of the images, and therefore improved retention of texture in the averaged image 70, may be achieved by selecting comparison images I_{Comp} that are offset from the reference image I_{Ref} by less than the offset threshold. Any affine frame offsets (typically a translation and/or rotation) the selected images 25 may be effectively compensated for by the image registration performed in process S40 of FIG. 4 such that the registered images are highly correlated.

The amount of texture in the averaged image 70 may be quantified using an algorithm as described in the article “Detection of Textured Areas in Images Using a Disorganization Indicator Based on Component Counts” by R. Bergman et al., J. Electronic Imaging. 17. 043003 (2008), the contents of which are incorporated herein by reference in their entirety. The texture detector presented in this article is based on the intuition that texture in a natural image is “disorganized”. The measure used to detect texture examines the structure of local regions of the image. This structural approach allows both structured and unstructured texture at many scales to be detected. Furthermore, it distinguishes between edges and texture, and also between texture and noise. Automatic detection results are shown in the article to match human classification of corresponding image areas. The amounts of texture in the averaged image 70 and the reference averaged image 75 may be compared by comparing the areas of these images that are designated as ‘texture’ by the algorithm.

The inventors have found that the use of interpolation to register the selected images 25 with respect to each other in process S40 of FIG. 4 tends to remove at least some of the texture that is present in the images acquired by the ophthalmic imaging device 10, and that more of the original texture may be retained in the averaged image 70 by registering the selected images 25 without interpolation, i.e. without interpolating between pixel values of any of the selected images 25 to register the images. The avoidance of interpolation in process S40 was found to not only improve the retention of this clinical data in the averaged image 70 but to also significantly improve the effectiveness of a machine learning (ML) denoising algorithm in distinguishing textural features from noise when the ML denoising algorithm has been trained using averaged images 70 that have been generated without interpolation.

Accordingly, the selected comparison images 25 may, as in the present example embodiment, be used by the processor 320 to generate the averaged image 70 of the region 30 by the processor 320 firstly registering the selected comparison images 25 with respect to one another. In this process, one of the selected images 25 may be selected as a base image, and each of the remaining selected images may be registered with respect to the base image, in turn, to generate a set of registered images. Where an image of the remaining images is being registered with respect to the base image, pixel values of a first image of the images in this pair of images are redistributed according to a geometric transformation between image coordinate systems of the images in the pair, i.e. pixel values of the first image are maintained but reassigned to different pixels in the first image in accordance with the geometric transformation that is required to register the images so that no interpolation is performed. The processor 320 then generates the averaged image 70 of the region 30 by averaging the registered images, for example such that the respective pixel value of each pixel in the averaged image 70 is a mean (e.g. an arithmetic mean) of the pixel values of correspondingly located pixels in the registered selected images 25 that correspond to respective common location in the region 30 of the retina 40. The processor 320 may alternatively generate the averaged image 70 by averaging the registered images such that the respective pixel value of each pixel in the averaged image 70 is a weighted average of the pixel values of correspondingly located pixels in the registered selected images 25 that correspond to respective common location in the region 30 of the retina 40.

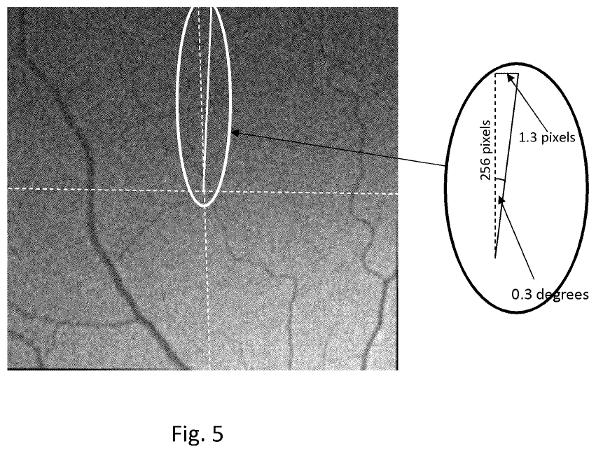

In case interpolation is avoided in process S40 of FIG. 4, the respective geometric transformation between the image coordinate systems of the images in each pair of the selected comparison images 25 consists of (i) a respective first translation, by a respective first integer numbers of pixels, along a first pixel array direction along which pixels of the selected comparison images 25 are arrayed, and/or (ii) a respective second translation, by a respective second integer numbers of pixels, along a second pixel array direction along which the pixels of the selected comparison images 25 are arrayed, the second direction being orthogonal to the first direction. Thus, no rotation of either image in the pair of images being registered occurs. The use of this constraint in the registration process requires the rotational offset threshold Θ used in process S20 of FIG. 4 to be small enough for an image rotation, which would be required to compensate for the rotational offset Δϕ between the images in the pair, to result in a displacement at an edge of the image frame which is smaller than a resolution of the ophthalmic imaging device 10. For example, in the case of a FAF imaging device, the resolution is typically limited by the point spread function of the device so the rotational offset threshold Θ would be set such that rotations required for registration, which produce displacements along an edge of the image frame that are smaller than the width of the point spread function, would be smaller than Θ (in which case the comparison image in the pair of images would be selected in S30 of FIG. 4), while rotations required for registration, which produce displacements along an edge of the image frame that are greater than the width of the point spread function, would be greater than Θ (in which case the comparison image in the pair of images would not be selected in S30 of FIG. 4). By way of an illustrative example, where the images in the sequence are 512×512 pixels in size and the point spread function for the region 30 is 1.5 pixels wide, as illustrated in FIG. 5, then setting Θ=0.3 degrees would require the maximal displacement caused by the rotation for registration (at an edge of the image frame) to be tan(0.3)·256=1.3 pixels, which is smaller than the width of the point spread function.

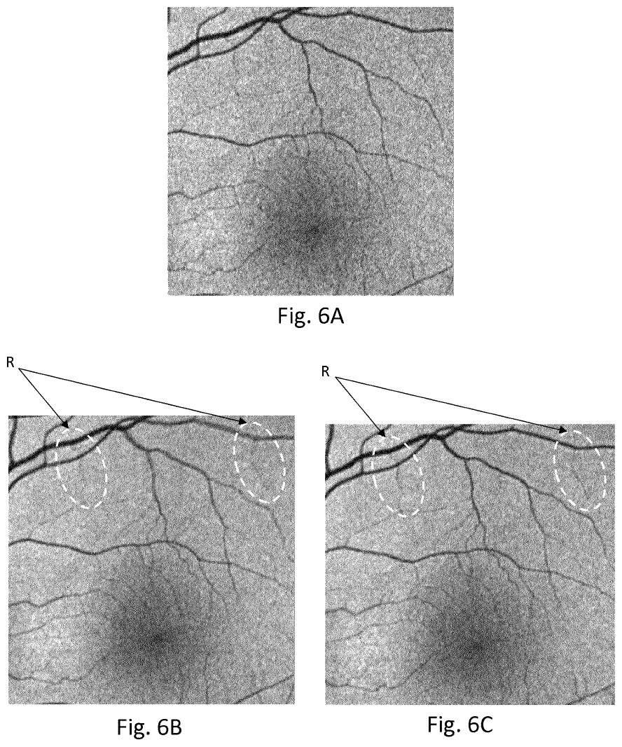

An averaged image, which has been obtained by averaging the example FAF image shown in FIG. 6A with another FAF image which is rotationally offset from the image of FIG. 6A by 4.5 degrees, is shown in FIG. 6B. For comparison, another averaged image, which has been obtained by averaging the FAF image shown in FIG. 6A with another FAF image that has a negligible rotational offset from the image of FIG. 6A, is shown in FIG. 6C. As can be appreciated from a comparison of FIGS. 6B and 6C, the averaged image of FIG. 6C shows anatomical detail in the highlighted regions R that is absent in the averaged image of FIG. 6B.

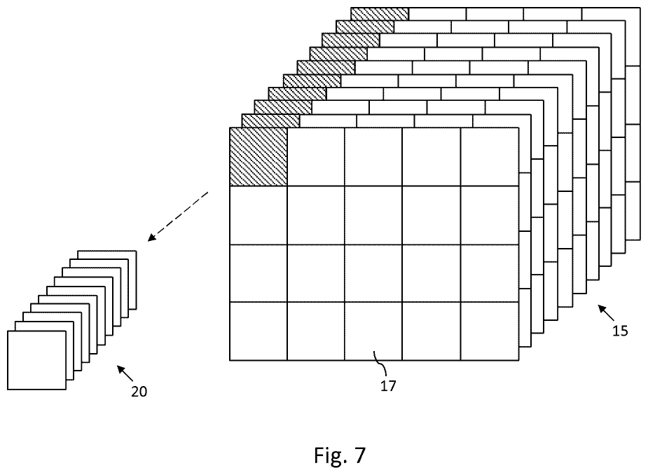

In some cases, a high proportion of the images in the sequence 20 may have intra-frame warping and/or localised regions of micro-warping (where there is micro-misalignment between imaged retinal features) of the kind noted above. In these cases, a relatively low proportion of the images in the sequence of images 20 may be selected in process S30 of FIG. 4 for averaging to generate the averaged image 70, and noise suppression in the averaged image 70 may consequently be less effective. In such cases, it may be beneficial to adapt the process of FIG. 4 by including, as illustrated schematically in FIG. 7, a preliminary process (before S10) of generating each image of the sequence of images 20 by segmenting a respective image of a second sequence of images 15 of a second region of the retina 40, such that the image of the region 30 is a segment 17 of the respective image of the second sequence of images 15. The sequence of images 20 may thus be a sequence of image segments of a second sequence of images 15 acquired by the ophthalmic imaging device 10 imaging a larger region of the retina 40 that includes the region 30, where each image segment 17 has a respective location within the respective image of the second sequence of images 15 which is the same as the respective location of every other image segment within the respective image of the second sequence of images 15. Limiting the subsequent image processing operations in the process of FIG. 4 to segments of the second sequence of images 15 may result in a relatively high proportion of images segments being selected in process S30 for averaging to generate the averaged image 70, and consequently less noise in the averaged image 70 than in a case where the images in the second sequence of images 15 are processed as a whole in accordance with the process described above with reference to FIG. 4. Since an averaged image 70 based on such a selection of the image segments, although less noisy, will cover only part of the region of the retina 40 that is spanned by the images in the second sequence of images 15, a plurality of other correspondingly located segments 17 of images in the second sequence of images 15 may be processed in the same way to generate additional averaged images that collectively cover more of the second region of the retina 40.

Each image of the second sequence of images 15 may be segmented in the same way into a one- or two-dimensional array of rectangular image tiles (e.g. a two-dimensional array of square image tiles, as illustrated in FIG. 7), and each set of correspondingly located image tiles from the segmented images may be processed in accordance with the process described above with reference to FIG. 4 to generate a corresponding averaged image tile. The averaged image tiles may then be combined to generate an averaged image of the second region of the retina, which may exhibit varying levels of noise suppression among the component averaged image tiles. The averaged image tiles may also be useful for training a machine learning denoising algorithm to more effectively distinguish between noise and retinal structures, and thus be able to produce de-noised retinal images that retain more of the texture present in the originally acquired images, as described below. It should be noted that the two-dimensional orthogonal array of square image segments 17 in FIG. 7 is given by way of an example only, and that the images segments 17 need not be of the same size or shape, and need not be arranged in a regular array.

### Second Example Embodiment

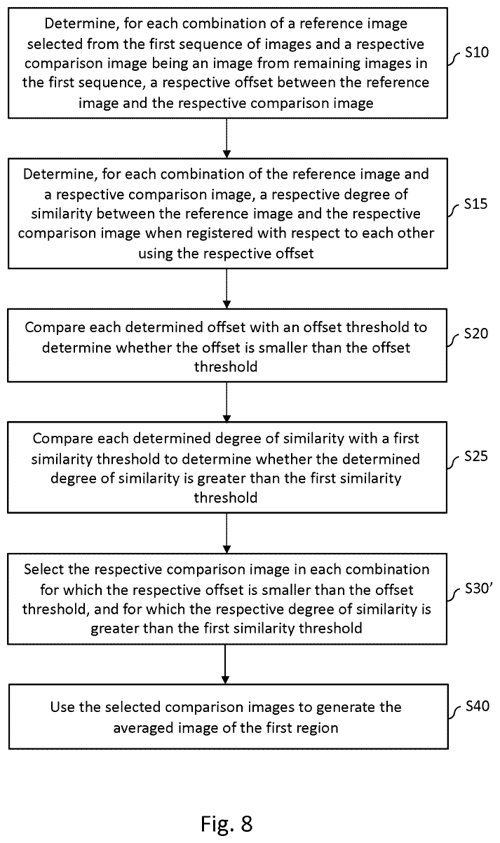

FIG. 8 is a flow diagram illustrating a method according to a second example embodiment, by which the data processing apparatus 60 may process the sequence of images 20 to generate an averaged image 70 of the region 30.

Processes S10, S20 and S40 in FIG. 8 are the same as those in FIG. 4 and will not be described again here. The method of the second example embodiment differs from the method of the first example embodiment by comprising additional processes S15 and S25, as well as a modified form of process S30 (labelled S30′ in FIG. 8), and these processes will now be described.

In process S15 of FIG. 8, the processor 320 determines, for each combination of the reference image I_{Ref} and a respective comparison image I_{Comp}, a respective degree of similarity between the reference image I_{Ref} and the respective comparison image I_{Comp} when registered with respect to each other using the respective offset(s), for example the respective translational offset t and the respective rotational offset Δϕ of the first example embodiment described above. The respective translational offset t between the reference image I_{Ref} and the respective comparison image I_{Comp} in each combination may, as in the first example embodiment, be determined by calculating a cross-correlation between the reference image I_{Ref} and the respective comparison image I_{Comp}. In this case, the respective degree of similarity between the reference image I_{Ref} and the respective comparison image I_{Comp}, when registered with respect to each other using the respective translational offset t, may be determined by determining a maximum value of the calculated cross-correlation. It is noted that the order of processes S10 and S15 may be reversed in some example embodiments. Accordingly, once the processor 320 has calculated the cross-correlation between the reference image I_{Ref} and the respective comparison image I_{Comp}, the processor 320 may determine the maximum value of the calculated cross-correlation before determining the translational (x-y) offset corresponding to that maximum value. It should be noted that the respective degree of similarity between the reference image I_{Ref} and the respective comparison image I_{Comp}, when registered with respect to each other using the respective offset(s), need not be derived from a calculated cross-correlation between the images and may instead be obtained by calculating a sum of squared differences or mutual information based on the images, for example.

In process S25 of FIG. 7, the processor 320 compares each degree of similarity determined in process S15 with a first similarity threshold to determine whether the determined degree of similarity is greater than the first similarity threshold. It is noted that the order of processes S20 and S25 may be reversed in some example embodiments.

In process S30′ of FIG. 8, the processor 320 selects the respective comparison image I_{Comp} in each combination for which the reference image I_{Ref} and the respective comparison image I_{Comp} have been determined to have a respective offset therebetween which is smaller than the offset threshold (i.e. each combination which satisfies the condition in S30 of FIG. 4), and a respective degree of similarity, when registered with respect to each other using the respective offset, which is greater than the first similarity threshold. The latter additional selection criterion helps avoid comparison images I_{Comp} that have intra-frame non-affine warping and/or comparison images I_{Comp} having micro-warping (i.e. where misalignment of retinal features is limited to one or more localised regions) being selected for averaging to generate the averaged image 70, as such images tend to be relatively poorly correlated to the reference image I_{Ref} even when the offset(s) between them and the reference image I_{Ref} are determined to be smaller than the offset threshold(s). Additional processes S15 and S25, and modified process S30′ of FIG. 8, may thus assist in avoiding loss of valuable clinical data such as texture in the averaged image 70. The first similarity threshold may be set by trial and error, observing the effect of adjustments of the first similarity threshold on the amount of texture present in the averaged image 70, for example.

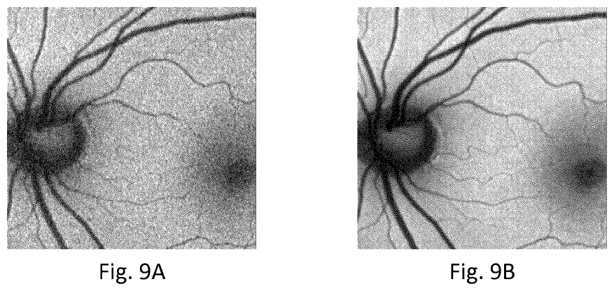

FIG. 9A shows an example FAF image acquired by the ophthalmic imaging device 10. For comparison, FIG. 9B shows an example of the averaged image 70, which has been obtained by averaging 10 FAF images, including the FAF image of FIG. 9A, that have been selected from a sequence of FAF images in accordance with the method described herein with reference to FIG. 8. FIG. 9B shows the averaged image to have less noise than the FAF image of FIG. 9A whilst retaining much of the texture of the FAF image of FIG. 9A (most apparent in the light grey regions of the image).

Referring again to FIG. 8, in process S25, the processor 320 may also compare each determined degree of similarity with a second similarity threshold to determine whether the determined degree of similarity is smaller than the second similarity threshold, the second similarity threshold being greater than the first similarity threshold. In this case, the processor 320 may perform a modified version of process S30′, by selecting the respective comparison image I_{Comp} in each combination for which the reference image I_{Ref} and the respective comparison image I_{Comp} have been determined to have a respective offset therebetween which is smaller than the offset threshold, and a respective degree of similarity, when the reference image I_{Ref} and the respective comparison image I_{Comp} are registered with respect to each other using the respective offset, which is greater than the first similarity threshold and smaller than the second similarity threshold. Comparison images may thus be selected for averaging to produce the averaged image 70 using the same criteria as in the first example embodiment, and the additional criterion that the degree of similarity is within a predefined range of values, i.e. between the first similarity threshold and the second similarity threshold. The exclusion from the selection of comparison images that yield excessively high determined degrees of similarity (as determined by comparison with an appropriate value for the second similarity threshold set by trial and error), as well as those that yield insufficiently high determined degrees of similarity (as determined by comparison with an appropriate value for the first similarity threshold set by trial and error), was found by the inventor to improve the retention of texture in the averaged image 70. Without wishing to be bound by theory, the reason for this may be understood as follows. There is a certain amount of information in any imaged region on the retina, and each single image can be considered to capture only a proportion of that information space, due to factors such as subtle variations in lighting, changes in the scanning system (such as line start positioning and subtle changes in related optical paths) and also other complex effects as to the way light scattering works from layers in the retina. Statistically, images of the region that differ slightly in information content will tend to produce an averaged image that overall reassembles a greater proportion of the total amount of information available than either of the individual images. From another perspective, very highly correlated images with exactly the same information content would not produce an averaged image with more of the information than either individual image but will still contain noise

### Third Example Embodiment

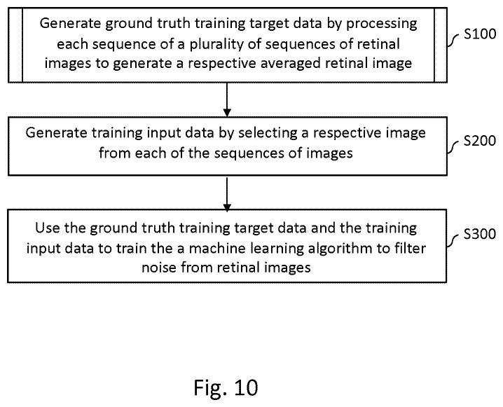

FIG. 10 is a flow diagram illustrating a method according to a third example embodiment, by which the data processing apparatus 60 trains a machine learning (ML) algorithm to filter noise from retinal images. The ML algorithm may be any kind of supervised ML algorithm known to those versed in the art which is suitable for removing noise of one or more different types from FAF images or any other kind of retinal images, once it has been trained to perform this task using labelled training data. The ML algorithm may comprise a convolutional neural network (CNN), as in the present example embodiment. The article titled “Image De-Noising With Machine Learning: A Review” by R. S. Thakur et al., IEEE Access, Vol. 9, pages 93338-93363 (2021), the contents of which are incorporated herein by reference in their entirety, describes various state-of-the-art machine-learning-based image de-noisers like dictionary learning models, convolutional neural networks and generative adversarial networks for a range of noises like Gaussian, Impulse, Poisson, Mixed and Real-World noises. The ML algorithm may be of any of the different kinds described in this article or otherwise known to those versed in the art.

In process S100 of FIG. 10, the processor 320 of the data processing apparatus 60 generates ground truth training target data by processing each sequence 20 of a plurality of sequences of retinal images as described in any of the foregoing example embodiments or their variants to generate a respective averaged retinal image.

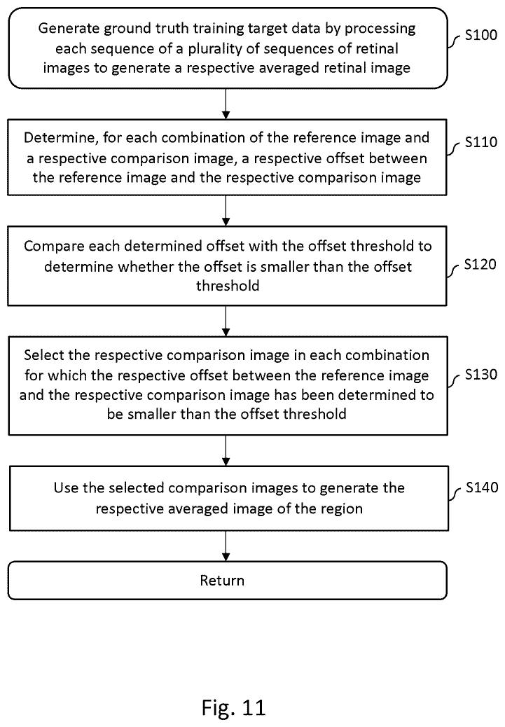

FIG. 11 is a flow diagram summarising the method by which the processor 320 generates each of the averaged retinal images from the respective sequence of retinal images in the present example embodiment.

In process S110 of FIG. 11, the processor 320 determines, for each combination of the reference image I_{Ref} and a respective comparison image I_{Comp} being an image from remaining images in the sequence 20, a respective offset between the reference image I_{Ref} and the respective comparison image I_{Comp}.

In process S120 of FIG. 11, the processor 320 compares each determined offset with the offset threshold to determine whether the offset is smaller than the offset threshold.

In process S130 of FIG. 11, the processor 320 selects the respective comparison image I_{Comp} in each combination for which the respective offset between the reference image I_{Ref} and the respective comparison image I_{Comp} has been determined to be smaller than the offset threshold.

In process S140 of FIG. 11, the processor 320 uses the selected comparison images 25 to generate the respective averaged image 70 of the region 30.

As explained above, the offset threshold is such that, where the sequence 20 comprises at least one image which is offset from the reference image I_{Ref} by an offset greater than the threshold, and images that are offset from the reference image I_{Ref} by respective offsets that are smaller than the threshold, the averaged image 70 shows more texture which is indicative of a structure in the first region 30 of the retina 40 than a reference averaged image generated from the images in the sequence of images 20.

Referring again to FIG. 10, in process S200, the processor 320 generates training input data by selecting a respective image from each of the sequences of images.

Then, in process S300 of FIG. 10, the processor 320 uses the ground truth training target data and the training input data to train the ML algorithm to filter noise from retinal images using techniques well known to those versed in the art.

Some of the example embodiments described above are summarised in following numbered clauses E1 to E14.

E1. A data processing apparatus 60 arranged to process a first sequence of images 20 of a region 30 of a retina 40 of an eye 50 to generate an averaged image 70 of the region 30, the data processing apparatus 60 comprising at least one processor 320 and at least one memory 340 storing computer-readable instructions that, when executed by the at least one processor 320, cause the at least one processor 320 to: determine, for each combination of a reference image I_{Ref} selected from the first sequence of images 20 and a respective comparison image I_{Comp} being an image from remaining images in the first sequence of images 20, a respective offset between the reference image I_{Ref} and the respective comparison image I_{Comp};
compare each determined offset with an offset threshold to determine whether the offset is smaller than the offset threshold;
select the respective comparison image I_{Comp} in each combination for which the respective offset between the reference image I_{Ref} and the respective comparison image I_{Comp} has been determined to be smaller than the offset threshold; and
use the selected comparison images 25 to generate the averaged image 70 of the region 30,
wherein the offset threshold is such that, where the first sequence of images 20 comprises at least one image which is offset from the reference image I_{Ref} by an offset greater than the threshold, and images that are offset from the reference image I_{Ref} by respective offsets that are smaller than the threshold, the averaged image 70 shows more texture which is indicative of a structure in the first region 30 of the retina 40 than a reference averaged image generated from the images in the first sequence of images 20.

E2. The data processing apparatus 60 of E1, wherein
the respective offset determined for each combination of the reference image I_{Ref} and the respective comparison image I_{Comp} comprises a translational offset, and
the computer-readable instructions, when executed by the at least one processor 320, cause the at least one processor 320 to:
compare each determined offset with the offset threshold to determine whether the offset is smaller than the offset threshold by comparing each translational offset with a translational offset threshold to determine whether the translational offset is smaller than the translational offset threshold; and
select the respective comparison image I_{Comp} in each combination for which the respective offset between the reference image I_{Ref} and the respective comparison image I_{Comp} has been determined to be smaller than the offset threshold by selecting the respective comparison image I_{Comp} in each combination for which the respective translational offset has been determined to be smaller than the translational offset threshold.

E3. The data processing apparatus 60 of E2, wherein the computer-readable instructions, when executed by the at least one processor 320, cause the at least one processor 320 to determine the respective translational offset between the reference image I_{Ref} and the respective comparison image I_{Comp} in each combination by one of:
calculating a cross-correlation using the reference image I_{Ref} and the respective comparison image I_{Comp}; and
calculating an inverse Fourier transform of a normalized cross-power spectrum calculated using the reference image I_{Ref} and the respective comparison image I_{Comp}.

E4. The data processing apparatus 60 of any of E1 to E3, wherein
the respective offset determined for each combination of the reference image I_{Ref} and the respective comparison image I_{Comp} comprises a rotational offset, and
the computer-readable instructions, when executed by the at least one processor 320, cause the at least one processor 320 to:
compare each determined offset with the offset threshold to determine whether the offset is smaller than the offset threshold by comparing each rotational offset with a rotational offset threshold to determine whether the rotational offset is smaller than the rotational offset threshold; and
select the respective comparison image I_{Comp} in each combination for which the respective offset between the reference image I_{Ref} and the respective comparison image I_{Comp} has been determined to be smaller than the offset threshold by selecting the respective comparison image I_{Comp} in each combination for which the respective rotational offset has been determined to be smaller than the rotational offset threshold.

E5. The data processing apparatus 60 of E4, wherein the computer-readable instructions, when executed by the at least one processor 320, cause the at least one processor 320 to determine the respective rotational offset between the reference image I_{Ref} and the respective comparison image I_{Comp} in each combination by one of:
calculating cross-correlations using rotated versions of the respective comparison image I_{Comp}; and
calculating an inverse Fourier transform of a normalized cross-power spectrum calculated using polar transformations of the reference image I_{Ref} and the respective comparison.

E6. The data processing apparatus 60 of any of E1 to E5, wherein the computer-readable instructions, when executed by the at least one processor 320, cause the at least one processor 320 to use the selected comparison images 25 to generate the averaged image of the first region by:
registering the selected comparison images 25 with respect to one another, wherein registering each pair of the selected comparison images 25 comprises redistributing pixel values of one of the images in the pair according to a respective geometric transformation between image coordinate systems of the images in the pair; and
generating the averaged image 70 of the first region by averaging the registered images.

E7. The data processing apparatus 60 of E6, wherein the respective geometric transformation between the image coordinate systems of the images in each pair of the selected comparison images 25 consists of at least one of:
a respective first translation, by a respective first integer numbers of pixels, along a first pixel array direction along which pixels of the selected comparison images 25 are arrayed; and
a respective second translation, by a respective second integer numbers of pixels, along a second pixel array direction along which the pixels of the selected comparison images 25 are arrayed.

E8. The data processing apparatus 60 of any of E1 to E7, wherein the computer-readable instructions, when executed by the at least one processor 320, further cause the at least one processor 320 to:
determine, for each combination of the reference image I_{Ref} and a respective comparison image I_{Comp}, a respective degree of similarity between the reference image I_{Ref} and the respective comparison image I_{Comp} when registered with respect to each other using the respective offset; and
compare each determined degree of similarity with a first similarity threshold to determine whether the determined degree of similarity is greater than the first similarity threshold,
wherein the computer-readable instructions, when executed by the at least one processor 320, cause the at least one processor 320 to select the respective comparison image I_{Comp} in each combination for which the reference image I_{Ref} and the respective comparison image I_{Comp} have been determined to have a respective offset therebetween which is smaller than the offset threshold, and a respective degree of similarity, when registered with respect to each other using the respective offset, which is greater than the first similarity threshold.

E9. The data processing apparatus 60 of E8, wherein
the computer-readable instructions, when executed by the at least one processor 320, further cause the at least one processor 320 to compare each determined degree of similarity with a second similarity threshold to determine whether the determined degree of similarity is smaller than the second similarity threshold, the second similarity threshold being greater than the first similarity threshold, and
the computer-readable instructions, when executed by the at least one processor 320, cause the at least one processor 320 to select the respective comparison image I_{Comp} in each combination for which the reference image I_{Ref} and the respective comparison image I_{Comp} have been determined to have a respective offset therebetween which is smaller than the offset threshold, and a respective degree of similarity, when registered with respect to each other using the respective offset, which is greater than the first similarity threshold and smaller than the second similarity threshold.

E10. The data processing apparatus 60 of E8 when dependent on E2, or on E9 when dependent on E2, wherein the computer-readable instructions, when executed by the at least one processor 320, cause the at least one processor 320 to:
determine the respective translational offset between the reference image I_{Ref} and the respective comparison image I_{Comp} in each combination by calculating a cross-correlation using the reference image I_{Ref} and the respective comparison image I_{Comp}, and
determine the respective degree of similarity between the reference image I_{Ref} and the respective comparison image I_{Comp} when registered with respect to each other using the respective translational offset, by determining a maximum value of the calculated cross-correlation.

E11. The data processing apparatus 60 of any of E1 to E10, wherein the computer-readable instructions, when executed by the at least one processor 320, further cause the at least one processor 320 to generate each image of the first sequence of images 20 by segmenting a respective image of a second sequence of images of a second region of the retina 40, such that the image of the first region 30 is a segment of the respective image of the second sequence of images.

E12. The data processing apparatus 60 of any of E1 to E11, wherein the first sequence of images 20 comprises a sequence of autofluorescence images of the first region 30 of the retina 40 of the eye 50.

E13. The data processing apparatus 60 of any of E1 to E12, wherein the computer-readable instructions, when executed by the at least one processor 320, further cause the at least one processor 320 to train a machine learning algorithm to filter noise from retinal images, by:
generating ground truth training target data by processing each sequence 20 of a plurality of sequences of retinal images to generate respective averaged retinal images, each of the averaged retinal images being generated by:
   determining, for each combination of the reference image I_{Ref} and a respective comparison image I_{Comp} being an image from remaining images in the sequence 20, a respective offset between the reference image I_{Ref} and the respective comparison image I_{Comp};
   comparing each determined offset with the offset threshold to determine whether the offset is smaller than the offset threshold;
   selecting the respective comparison image I_{Comp} in each combination for which the respective offset between the reference image I_{Ref} and the respective comparison image I_{Comp} has been determined to be smaller than the offset threshold; and
   using the selected comparison images 25 to generate the respective averaged image 70 of the region 30,
   wherein the offset threshold is such that, where the sequence 20 comprises at least one image which is offset from the reference image I_{Ref} by an offset greater than the threshold, and images that are offset from the reference image I_{Ref} by respective offsets that are smaller than the threshold, the averaged image 70 shows more texture which is indicative of a structure in the first region 30 of the retina 40 than a reference averaged image generated from the images in the sequence of images 20;
generating training input data by selecting a respective image from each of the sequences of images; and
using the ground truth training target data and the training input data to train the machine learning algorithm to filter noise from retinal images.

E14. An ophthalmic imaging system 100 comprising:
an ophthalmic imaging device 10 arranged to acquire a sequence of images 20 of a region 30 of a retina 40 of an eye 50; and
a data processing apparatus 60 according to any of E1 to E13, which is arranged to process the sequence of images 20 acquired by the ophthalmic imaging device 10 to generate an averaged image 70 of the region 30 of the retina 40.

In the foregoing description, example aspects are described with reference to several example embodiments. Accordingly, the specification should be regarded as illustrative, rather than restrictive. Similarly, the figures illustrated in the drawings, which highlight the functionality and advantages of the example embodiments, are presented for example purposes only. The architecture of the example embodiments is sufficiently flexible and configurable, such that it may be utilized in ways other than those shown in the accompanying figures.

Some aspects of the examples presented herein may be provided as a computer program, or software, such as one or more programs having instructions or sequences of instructions, included or stored in an article of manufacture such as a machine-accessible or machine-readable medium, an instruction store, or computer-readable storage device, each of which can be non-transitory, in one example embodiment. The program or instructions on the non-transitory machine-accessible medium, machine-readable medium, instruction store, or computer-readable storage device, may be used to program a computer system or other electronic device. The machine- or computer-readable medium, instruction store, and storage device may include, but are not limited to, floppy diskettes, optical disks, and magneto-optical disks or other types of media/machine-readable medium/instruction store/storage device suitable for storing or transmitting electronic instructions. The techniques described herein are not limited to any particular software configuration. They may find applicability in any computing or processing environment. The terms “computer-readable”, “machine-accessible medium”, “machine-readable medium”, “instruction store”, and “computer-readable storage device” used herein shall include any medium that is capable of storing, encoding, or transmitting instructions or a sequence of instructions for execution by the machine, computer, or computer processor and that causes the machine/computer/computer processor to perform any one of the methods described herein. Furthermore, it is common in the art to speak of software, in one form or another (e.g., program, procedure, process, application, module, unit, logic, and so on), as taking an action or causing a result. Such expressions are merely a shorthand way of stating that the execution of the software by a processing system causes the processor to perform an action to produce a result.

Some or all of the functionality of the data processing apparatus 60 may also be implemented by the preparation of application-specific integrated circuits, field-programmable gate arrays, or by interconnecting an appropriate network of conventional component circuits.

A computer program product may be provided in the form of a storage medium or media, instruction store(s), or storage device(s), having instructions stored thereon or therein which can be used to control, or cause, a computer or computer processor to perform any of the procedures of the example embodiments described herein. The storage medium/instruction store/storage device may include, by example and without limitation, an optical disc, a ROM, a RAM, an EPROM, an EEPROM, a DRAM, a VRAM, a flash memory, a flash card, a magnetic card, an optical card, nanosystems, a molecular memory integrated circuit, a RAID, remote data storage/archive/warehousing, and/or any other type of device suitable for storing instructions and/or data.

Stored on any one of the computer-readable medium or media, instruction store(s), or storage device(s), some implementations include software for controlling both the hardware of the system and for enabling the system or microprocessor to interact with a human user or other mechanism utilizing the results of the example embodiments described herein. Such software may include without limitation device drivers, operating systems, and user applications. Ultimately, such computer-readable media or storage device(s) further include software for performing example aspects of the invention, as described above.

Included in the programming and/or software of the system are software modules for implementing the procedures described herein. In some example embodiments herein, a module includes software, although in other example embodiments herein, a module includes hardware, or a combination of hardware and software.

While various example embodiments of the present invention have been described above, it should be understood that they have been presented by way of example, and not limitation. It will be apparent to persons skilled in the relevant art(s) that various changes in form and detail can be made therein. Thus, the present invention should not be limited by any of the above-described example embodiments, but should be defined only in accordance with the following claims and their equivalents.

Further, the purpose of the Abstract is to enable the Patent Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the example embodiments presented herein in any way. It is also to be understood that any procedures recited in the claims need not be performed in the order presented.

While this specification contains many specific embodiment details, these should not be construed as limitations on the scope of any inventions or of what may be claimed, but rather as descriptions of features specific to particular embodiments described herein. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

In certain circumstances, multitasking and parallel processing may be advantageous. Moreover, the separation of various components in the embodiments described above should not be understood as requiring such separation in all embodiments, and it should be understood that the described program components and systems can generally be integrated together in a single software product or packaged into multiple software products.

Having now described some illustrative embodiments and embodiments, it is apparent that the foregoing is illustrative and not limiting, having been presented by way of example. In particular, although many of the examples presented herein involve specific combinations of apparatus or software elements, those elements may be combined in other ways to accomplish the same objectives. Acts, elements and features discussed only in connection with one embodiment are not intended to be excluded from a similar role in other embodiments or embodiments.

1. A computer-implemented method of processing a first sequence of images of a first region of a retina of an eye to generate an averaged image of the first region, the method comprising:
determining, for each combination of a reference image (I_{Ref}) selected from the first sequence of images and a respective comparison image (I_{Comp}) being an image from remaining images in the first sequence, a respective offset (t; Δφ) between the reference image (I_{Ref}) and the respective comparison image (I_{Comp});
comparing each determined offset (t; Δϕ) with an offset threshold (T; Θ) to determine whether the offset (t; Δϕ) is smaller than the offset threshold (T; Θ);
selecting the respective comparison image (I_{Comp}) in each combination for which the respective offset (t) between the reference image (I_{Ref}) and the respective comparison image (I_{Comp}) has been determined to be smaller than the offset threshold (T; Θ); and
using the selected comparison images to generate the averaged image of the first region,
wherein the offset threshold (T; Θ) is such that, where the first sequence of images comprises at least one image which is offset from the reference image (I_{Ref}) by an offset (t) greater than the threshold (T), and images that are offset from the reference image (I_{Ref}) by respective offsets (t) that are smaller than the threshold (T), the averaged image shows more texture which is indicative of a structure in the first region of the retina than a reference averaged image generated from the images in the first sequence of images.

2. The computer-implemented method of claim 1, wherein
the respective offset (t; Δφ) determined for each combination of the reference image (I_{Ref}) and the respective comparison image (I_{Comp}) comprises a translational offset (t),
the comparing comprises comparing each translational offset (t) with a translational offset threshold (T) to determine whether the translational offset (t) is smaller than the translational offset threshold (T), and
the selecting comprises selecting the respective comparison image (I_{Comp}) in each combination for which the respective translational offset (t) has been determined to be smaller than the translational offset threshold (T).

3. The computer-implemented method of claim 2, wherein the respective translational offset (t) between the reference image (I_{Ref}) and the respective comparison image (I_{Comp}) in each combination is determined by one of:
calculating a cross-correlation using the reference image (I_{Ref}) and the respective comparison image (I_{Comp}); and
calculating an inverse Fourier transform of a normalized cross-power spectrum calculated using the reference image (I_{Ref}) and the respective comparison image (I_{Comp}).

4. The computer-implemented method of claim 1, wherein:
the respective offset (t; Δϕ) determined for each combination of the reference image (I_{Ref}) and the respective comparison image (I_{Comp}) comprises a rotational offset (Δφ),
the comparing comprises comparing each rotational offset (Δφ) with a rotational offset threshold (Θ) to determine whether the rotational offset (Δφ) is smaller than the rotational offset threshold (Θ), and
the selecting comprises selecting the respective comparison image (I_{Comp}) in each combination for which the respective rotational offset (Δφ) has been determined to be smaller than the rotational offset threshold (Θ).

5. The computer-implemented method of claim 4, wherein the respective rotational offset (Δφ) between the reference image (I_{Ref}) and the respective comparison image (I_{Comp}) in each combination is determined by one of:
calculating cross-correlations using rotated versions of the respective comparison image (I_{Comp}); and
calculating an inverse Fourier transform of a normalized cross-power spectrum calculated using polar transformations of the reference image (I_{Ref}) and the respective comparison image (I_{Comp}).

6. The computer-implemented method of claim 1, wherein the selected comparison images are used to generate the averaged image of the first region by:
registering the selected comparison images with respect to one another, wherein registering each pair of the selected comparison images comprises redistributing pixel values of one of the images in the pair according to a respective geometric transformation between image coordinate systems of the images in the pair; and
generating the averaged image of the first region by averaging the registered images.

7. The computer-implemented method of claim 6, wherein the respective geometric transformation between the image coordinate systems of the images in each pair of the selected comparison images consists of at least one of:
a respective first translation, by a respective first integer numbers of pixels, along a first pixel array direction along which pixels of the selected comparison images are arrayed; and
a respective second translation, by a respective second integer numbers of pixels, along a second pixel array direction along which the pixels of the selected comparison images are arrayed.

8. The computer-implemented method of claim 2, further comprising:
determining, for each combination of the reference image (I_{Ref}) and a respective comparison image (I_{Comp}), a respective degree of similarity between the reference image (I_{Ref}) and the respective comparison image (I_{Comp}) when registered with respect to each other using the respective offset; and
comparing each determined degree of similarity with a first similarity threshold to determine whether the determined degree of similarity is greater than the first similarity threshold,
wherein the selecting comprises selecting the respective comparison image (I_{Comp}) in each combination for which the reference image (I_{Ref}) and the respective comparison image (I_{Comp}) have been determined to have a respective offset (t; Δϕ) therebetween which is smaller than the offset threshold (T; Θ), and a respective degree of similarity, when registered with respect to each other using the respective offset, which is greater than the first similarity threshold.

9. The computer-implemented method of claim 8, further comprising comparing each determined degree of similarity with a second similarity threshold to determine whether the determined degree of similarity is smaller than the second similarity threshold, the second similarity threshold being greater than the first similarity threshold, wherein the selecting comprises selecting the respective comparison image (I_{Comp}) in each combination for which the reference image (I_{Ref}) and the respective comparison image (I_{Comp}) have been determined to have a respective offset (t; Δϕ) therebetween which is smaller than the offset threshold (T; Θ), and a respective degree of similarity, when registered with respect to each other using the respective offset, which is greater than the first similarity threshold and smaller than the second similarity threshold.

10. The computer-implemented method of claim 8, wherein:
the respective translational offset (t) between the reference image (I_{Ref}) and the respective comparison image (I_{Comp}) in each combination is determined by calculating a cross-correlation using the reference image (I_{Ref}) and the respective comparison image (I_{Comp}), and
the respective degree of similarity between the reference image (I_{Ref}) and the respective comparison image (I_{Comp}), when registered with respect to each other using the respective translational offset (t), is determined by determining a maximum value of the calculated cross-correlation.

11. The computer-implemented method of claim 1, further comprising generating each image of the first sequence of images by segmenting a respective image of a second sequence of images of a second region of the retina, such that the image of the first region is a segment of the respective image of the second sequence of images.

12. The computer-implemented method of claim 1, wherein the first sequence of images comprises a sequence of autofluorescence images of the first region of the retina of the eye.

13. A computer-implemented method of training a machine learning algorithm to filter noise from retinal images, the method comprising:
generating ground truth training target data by processing each sequence of a plurality of sequences of retinal images to generate a respective averaged retinal image, wherein each averaged retinal image is generated in accordance with the computer-implemented method of any preceding claim;
generating training input data by selecting a respective image from each of the sequences of images; and
using the ground truth training target data and the training input data to train the machine learning algorithm to filter noise from retinal images.

14. A computer program comprising computer-readable instructions that, when executed by at least one processor, cause the at least one processor to execute a method according to claim 13.

15. A data processing apparatus arranged to process a sequence of images of a region of a retina of an eye to generate an averaged image of the region, the data processing apparatus comprising at least one processor and at least one memory storing computer-readable instructions that, when executed by the at least one processor, cause the at least one processor to:
determine, for each combination of a reference image (I_{Ref}) selected from the sequence of images and a respective comparison image (I_{Comp}) being an image from remaining images in the sequence, a respective offset (t; Δφ) between the reference image (I_{Ref}) and the respective comparison image (I_{Comp});
compare each determined offset (t; Δϕ) with an offset threshold (T; Θ) to determine whether the offset (t; Δϕ) is smaller than the offset threshold (T; Θ);
select the respective comparison image (I_{Comp}) in each combination for which the respective offset (T; Θ) between the reference image (I_{Ref}) and the respective comparison image (I_{Comp}) has been determined to be smaller than the offset threshold (T; Θ); and
use the selected comparison images to generate the averaged image of the region,
wherein the offset threshold (T; Θ) is such that, where the sequence of images comprises at least one image which is offset from the reference image (I_{Ref}) by an offset (t) greater than the threshold (T), and images that are offset from the reference image (I_{Ref}) by respective offsets (t) that are smaller than the threshold (T), the averaged image shows more texture which is indicative of a structure in the first region of the retina than a reference averaged image generated from the images in the sequence of images.

16. A computer program comprising computer-readable instructions that, when executed by at least one processor, cause the at least one processor to execute a method according to claim 1.
